(19) United States
(12) Patent Application Publication
(10) Pub. No.: US 2014/0296974 A1
(43) Pub. Date: Oct. 2, 2014

(54) SYSTEM AND METHOD FOR ASSISTING THE POSITIONING OF MEDICAL INSTRUMENTS

(71) Applicant: CORASSIST CARDIOVASCULAR LTD., Herzliya Pituach (IL)

(72) Inventors: Gideon Meyer-Brodnitz, Yokneam Moshava (IL); Michal Lipperman, Kfar Saba (IL); Lior Rosen, Tel Aviv (IL); Yaron Tal, Tel Mond (IL); Lea Lak, Tel Aviv (IL); Amir Sherman, Gedera (IL)

(21) Appl. No.: 14/355,815

(22) PCT Filed: Nov. 1, 2012
(86) PCT No.: PCT/IL2012/000368

§ 371 Date: May 1, 2014

Related U.S. Application Data
(60) Provisional application No. 61/554,012, filed on Nov. 1, 2011.

(51) Int. Cl. A61F 2/24; A61B 17/3213

(52) U.S. Cl. CPC A61F 2/2433; A61B 17/3213; A61B 2017/00247
USPC 623/2.11

(57) ABSTRACT

The present invention relates to a device, system and method for assisting the positioning of medical instruments. The system comprises a hollow tubular member such as a sheath or sleeve comprising a rotational indicator element. The system further comprises an elongated rod having a blade on its distal end and comprising a rotational indicator element (aligned with the blade orientation) and a stopper close to its proximal end.

### FIELD OF THE INVENTION

The present invention relates to a device and method for use in assisting the delivery and correct placement or implantation of medical instruments and devices. More specifically, the delivery device and method disclosed herein are particularly suited for use in the implantation of intra-cardiac devices (such as those disclosed in co-owned, co-pending International Patent Application no. PCT/IL09/00988 (published as WO 2010/046895) and incorporated herein by reference.

### BACKGROUND OF THE INVENTION

Insertion of medical instruments in the human body for various medical treatments requires a great deal of caution and preciseness in order not to damage the target organs as well as the tissues and organs surrounding them, and also ensuring that the medical instrument will be able to properly perform its intended use.

When several implants, catheters, delivery system parts, etc. or other medical devices are inserted into the body they must be controlled and handled with extreme care and they must be inserted at the exact target location positioned and oriented in the most efficient manner. Positioning and orientation control are difficult for medical personnel to execute, whereas correct positioning and orientation is crucial for medical procedures such as cardiac procedures. The prior art methods have not yet provided an efficient way in assisting the correct positioning and orientation of implants and medical devices.

US 20080045989 relates to an endoscopic tool that utilizes a fiber optic system for illuminating and imaging ligaments or other tissue which are to be cut. Illumination and imaging is performed above a lateral opening at the distal end of a probe that is inserted into an incision point.

EP 1943974 relates to one or more markers or sensors (electromagnetic coils) that are positioned in the vasculature of a patient to facilitate determination of the location, configuration, and/or orientation of a vessel or certain aspects thereof (e.g., a branch vessel), as well as determining the location, configuration and/or orientation of endovascular devices prior to and during prosthesis deployment. This prior art invention may also be used to determine the relative position of portions of the vasculature and devices, generating an image of a virtual model of a portion of one or more vessels (e.g., branch vessels) or devices, and/or formation of one or more openings in a tubular prosthesis in situ to allow branch vessel perfusion, when the prosthesis is placed over one or more branch vessels in a patient (e.g., when an aortic abdominal artery stent-graft is fixed to the aorta superior to the renal artery ostia).

U.S. Pat. No. 5,217,024 relates to a medical device for removing tissue from a body cavity, includes a tubular shaped handle securely connected and in communication with an elongated hollow tubular probe. The probe includes a spherical tip and an open area with a notch. The open area includes a cutting edge for removing tissue. The handle has a visual and tactile indicator in the general shape of a raised arrow. The raised indicator is axially aligned with open area of the probe. Accordingly, the user can feel or see the raised indicator and know the exact orientation of the open area when the cutting edge of the device is in the body cavity.

When using conventional imaging modalities to try and view an implant/medical device for positioning in a specific organ, the outcome of the procedure is not clear. Echocardiography does not provide a clear view of the implant/medical device (especially when made of various steel components) and X-ray imaging does not provide a clear view of the organs. Therefore there is still a need for a method, system and device for clearly imaging the organs and simultaneously provide a clear and precise imaging for positioning the implants/devices within these organs.

None of the prior art methods provide efficient means for assisting the correct positioning and orientation of implants and medical devices.

It is therefore an object of the present invention to provide a method and means for determining a desired positioning and orientation of a medical device or implant to be inserted into the body.

It is a further object of the present invention to provide a method and means for inserting the medical device or implant in the determined positioning and orientation.

Other objects and advantages of the present invention will become apparent as the description proceeds.

### SUMMARY OF THE INVENTION

The present invention relates to a device, system and method for assisting the positioning of medical instruments. The system comprises a hollow tubular member such as a sheath or sleeve comprising a rotational indicator element. The system further comprises an elongated rod having a blade on its distal end and comprising a rotational indicator element (aligned with the blade orientation) and a stopper close to its proximal end.

In the present specification the “proximal end” refers to as the end closest to the user/medical personnel and the “distal end” refers to the end furthest from the user/medical personnel and closest to the target location in the patient's body.

The rod is adapted to enter the hollow tubular member, wherein the blade extends out distal to the hollow tubular member. Acoustic imaging is applied for observing the position and orientation of said elongated rod blade such that a user is able to distinguish between the narrow and broad sides of the blade when being turned. The imaging can also be performed using ultrasound, MRI, CT and other imaging methods.

The blade is rotated, positioned and oriented in a desired manner, with reference to anatomic data received from the acoustic imaging (or any of the alternative methods aforementioned) data.

The tubular member rotational indicator element is aligned with the rod indicator element. The rod is extracted and a device (e.g. implant or instrument) is inserted in the tubular member in a manner such that its orientation will be in accordance with the orientation of the tubular member rotational indicator element.

The present invention relates to a system for assisting the placement of medical instruments, wherein said system comprises:
a. an elongate rod having a proximal end and a distal end, and wherein a blade is situated at said distal end and a rotational indicator element is placed close to the proximal end;
b. a sheath adapted to receive said rod therein comprising a rotational indicator element thereon.

Preferably, the rod further comprises a mechanical stopper element situated close to the proximal end of said rod.

Preferably, the indicator element is placed on the mechanical stopper.

Preferably, the blade is elongated and substantially flat with a narrow thickness.

Preferably, the transverse cross-sectional width and area of the blade changes decreasingly, gradually from the proximal end of the blade to the distal end of the blade.

Preferably, the blade is provided in the form of a hollow frame.

Preferably, the blade comprises a series of hollow contours, with decreasing gradual changing volume of the contours from the proximal end to the distal end.

Preferably, the blade comprises a series of hollow contours, with decreasing gradual changing number of hollow contours from the proximal end to the distal end.

Preferably, the rod and sheath comprise one or more echo-opaque materials.

Preferably, the blade comprises bubbles wherein the bubbles material is selected from the group consisting of air, oil, and echo-detectable fluid.

Preferably, the system further comprising a dilator, wherein the rod and a dilator are combined in one tool, wherein the combined tool is adapted to be inserted through the sheath.

Preferably, the combined tool comprises a button at its proximal end such that when said button is pushed distally the blade is exposed at the combined tool distal end and when said button is pulled proximally the blade enters the combined tool.

The present invention relates to a system for assisting the placement of medical instruments, wherein said system comprises:
a. an elongate rod having a proximal end and a distal end and a rotational indicator element is placed close to the proximal end;
b. a dilator, wherein the rod and a dilator are combined in one tool, wherein the combined tool is adapted to be inserted through a sheath.
c. a sheath adapted to receive said combined tool therein comprising a rotational indicator element thereon.

Preferably, the combined tool further comprises two inflatable balloons near its distal end, opposite of each other such that when they are inflated they create a substantially flat mutual surface.

Preferably, the combined tool further comprises an inflatable balloon near its distal end.

Preferably, the inflatable balloon comprises an unsymmetrical shape when inflated.

Preferably, the dilator distal end has a conical structure and the conical structure is formed by two half cones; and wherein the combined tool comprises a button at its proximal end such that when said button is pushed distally one half cone moves distally and when said button is pulled proximally said one half cone moves proximally.

The present invention relates to a system for assisting the placement of medical instruments, wherein said system comprises:
a. an elongate flexible rod having a proximal end and a distal end, and wherein a blade is situated at said distal end and a rotational indicator element is placed close to the proximal end;
b. a delivery tool adapted to receive said rod therein comprising a rotational indicator element thereon.

Preferably, the delivery tool is selected from the group consisting of working channel, implant delivery system, instrument delivery system, catheter, and catheter with an additional distal dilator.

The present invention relates to a system for assisting the placement of medical instruments, wherein said system comprises:
a delivery tool comprising a rotational indicator element thereon and two inflatable balloons near its distal end, opposite of each other such that when they are inflated they create a substantially flat mutual surface aligned in the same rotational orientation as said indicator element.

The present invention relates to a method for assisting in the correct placement of a medical instrument within a target tissue of a mammalian subject, comprising the steps of:
a) inserting a hollow sleeve fitted with a rotational indicator element into said target tissue;
b) providing an elongated rod having a blade-like structure at its distal end and a rotatable indicator element and stopper towards its proximal end; adjusting the position of said stopper on said elongated rod such that a desired distance between said stopper and the distal tip of said rod is achieved;
c) inserting said elongated rod into the internal bore of said hollow sleeve until its stopper has reached the proximal end of said sleeve.
d) observing the position and orientation of said elongated rod by means of acoustic imaging;
e) rotating the elongated rod until the distal blade is in the desired rotational orientation;
f) adjusting the rotational indicator on the hollow sleeve until it is located parallel to the rotational indicator on the elongated rod;
g) removing said elongated rod; and
i) inserting a desired medical instrument into the internal bore of the hollow sleeve and rotating same until it is orientated in the desired rotational plane as indicated by the rotational indicator on said hollow sleeve.

Preferably, the medical instrument is an intra-cardiac device and wherein the rotational adjustment in step (e) is made such that the blade of the elongated rod becomes orientated within the plane defined by the space between the ventricular papillary muscles.

Preferably, the method further comprises a step after step e, comprising positioning the blade distal portion such that said blade distal portion is placed in the center of the ventricle,

Preferably, the placement of the distal blade in the center of the ventricle is implemented by viewing the acoustic trail casted by the pointer.

The present invention relates to a method for assisting in the correct placement of a medical instrument within a target tissue of a mammalian subject, comprising the steps of:
a) inserting a delivery tool fitted with a rotational indicator element into said target tissue;
b) providing an elongated flexible rod having a blade-like structure at its distal end and a rotatable indicator element towards its proximal end; inserting said elongated flexible rod into the internal bore of said delivery tool.
c) observing the position and orientation of said elongated flexible rod by means of acoustic imaging;
d) rotating the elongated flexible rod until the distal blade is in the desired rotational orientation;
e) adjusting the rotational indicator on the delivery tool until it is located parallel to the rotational indicator on the elongated flexible rod;
f) removing said elongated flexible rod; and
g) inserting a desired medical instrument into the internal bore of the delivery tool and rotating same until it is orientated in the desired rotational plane as indicated by the rotational indicator on said delivery tool.

Preferably, the delivery tool is selected from the group consisting of working channel, implant delivery system, instrument delivery system, catheter, and catheter with an additional distal dilator.

The present invention relates to a method for assisting in the correct placement of a medical instrument within a target tissue of a mammalian subject, comprising the steps of:
a) inserting a delivery tool into said target tissue;
b) providing one or more inflatable balloons at said delivery tool distal end and a rotatable indicator element towards its proximal end aligned with said one or more inflatable balloons structure;
c) inflating said one or more inflatable balloons;
d) observing the position and orientation of said one or more inflatable balloons by means of acoustic imaging;
e) rotating the elongated flexible rod until one or more inflatable balloons structure is in the desired rotational orientation;
f) inserting a desired medical instrument into the internal bore of the delivery tool and rotating same until it is orientated in the desired rotational plane as indicated by the rotational indicator on said delivery tool.

### BRIEF DESCRIPTION OF THE DRAWINGS

The present invention is illustrated by way of example in the accompanying drawings, in which similar references consistently indicate similar elements and in which:

FIGS. 1a-1d illustrate embodiments of the Papillary Muscle Pointer (PMP) and Trans Apical Sheath (TAS) of the present invention.

FIGS. 2a-2c illustrate embodiments of the PMP and TAS of the present invention.

FIG. 3 illustrates an embodiment of the PMP blade of the present invention.

FIGS. 4a-4b illustrate an example of the acoustic imaging according to an embodiment of the present invention.

FIGS. 5a-5c illustrate embodiments of the PMP blade of the present invention.

FIGS. 6a-6b illustrate an example of the acoustic imaging according to an embodiment of the present invention.

FIGS. 7a-7c illustrate embodiments of the PMP blade of the present invention.

FIGS. 8a-8b illustrate an example of the acoustic imaging according to an embodiment of the present invention.

FIGS. 9a-9b illustrate embodiments of the PMP blade of the present invention.

FIGS. 10a-10b illustrate embodiments of the PMP blade of the present invention.

FIGS. 11a-11b illustrate embodiments of an integrated Dilator and PMP of the present invention, while the PMP is in a retrieved (FIG. 11a) or exposed (FIG. 11b) condition.

FIGS. 12a-12b illustrate embodiments of the PMP of the present invention, while integrated with a balloon. FIG. 12a shows the PMP with the balloon deflated, while FIG. 12b shows the PMP with the balloon inflated.

FIGS. 13a-13d illustrate embodiments of PMP integrated with a balloon of the present invention, with various balloon shapes.

FIGS. 14a-14d illustrate cross sectional sections of the embodiments of FIGS. 13a-13d.

FIG. 14e illustrates cross sectional sections of various balloon shapes on the PMP.

FIGS. 15a-15c illustrate embodiments of the present invention while the PMP is introduced in a percutaneous approach through a delivery catheter.

FIGS. 16a-16b illustrate embodiments of the blade.

FIGS. 17a-17g illustrate different visual ultrasound images of the present invention inserted in the heart.

FIGS. 18a-18b illustrate pictorial views of the PMP and its elements.

### DETAILED DESCRIPTION OF THE INVENTION

The present invention provides an elongate echo-opaque instrument manufactured from echogenic materials such as biocompatible polymers (e.g. Acetal, resins, Delrin, FEP, PTFE, Silicon, to name a few) or biocompatible metals (such as stainless steel, gold, platinum, and cobalt-chromium alloys, to name a few), or a combination of both such metals and polymers, such that it is capable of projecting an acoustic shadow during echocardiography. By interpreting the acoustic shadow shapes (i.e. narrow, broad, short, long, circular, rectangular, etc.) that are observed, the insertion depth of the instrument device into the tissue as well as the orientation of the device, compared to relevant tissue structure or implant, can be assessed, thereby serving as a useful adjunct in various clinical procedures.

This effect can be used in order to indicate the rotational, longitudinal, and other spatial position of tools which are inserted into biological or other material transmission mediums (e.g. tissue or body fluids).

After in-vivo insertion of a designated pointer into a hollow organ or into a certain body lumen or into a biological or other material transmission medium (e.g. tissue or body fluids) through an introducer sheath, its orientation in said hollow organ may be changed (e.g. advanced inside or outside, rotated, etc.). Using an echocardiographic probe (for example a transesophageal, trans-thoracic, or trans-gastric probe), different sizes and shapes of acoustic shadow cast by said pointer may be observed. The shadow features are in direct consequence to the pointer depth, positioning and orientation. This effect can be used in order to determine the designated rotational, longitudinal, and other spatial position of tools which are to be inserted into the biological or other material transmission mediums, in order to ensure their precise positioning with reference to the surrounding anatomic landscape . . . . The narrow side of the pointer can point to a certain desired location This gives a medical personnel the ability to plan a tool insertion (e.g. a delivery tool for implantation) such that the item to be inserted will be in a correct direction. The acoustic trail, casted by the pointer, can also be viewed in echocardiography (for example), and further enables to view the exact location of the pointer's edge, with respect to the organ cavity in which it is located, e.g. the center of a ventricle. The placement in the center of the ventricle is implemented by viewing the location of the acoustic shadow trail and adjusting the position of the PMP such that the trail will originate from the center of the ventricle.

Optionally, the location positioning within the organ can be executed before the rotational orientation positioning.

One exemplary, particularly preferred, use of the device of the present invention is to assist in the correct orientation and implantation of an intra-cardiac device, more specifically, an intra-cardiac device for the treatment of diastolic heart failure, and particularly in the positioning of said device inside the heart ventricle. In this particularly preferred embodiment, the key purpose of the device is to indicate the orientation of the papillary muscles—in effect ‘pointing’ the direction of the space between the papillary muscles, preferably from the center of the ventricle's cavity, thereby assisting the operator to correctly align and insert other surgical instruments (such as dilators, introducers, delivery tools and the like) or implants (such as an implant treating the symptoms of diastolic heart failure) while a specific part of it is oriented to be situated into said space.

In another exemplary use, the device may be used in order to point the aortic valve or mitral valve, for example, to assist in the correct positioning (e.g. longitudinal, angular, etc.) of an aortic, mitral, pulmonary or tricuspid valve implant, delivered trans-apically or percutaneously.

It should be noted, however, that the scope of the present invention is not limited to the field of intra-cardiac devices alone. Rather, additional applications of the presently-disclosed device and method include (but are not limited to) other clinical procedures, such as those performed in the abdominal, urinary (including bladder), vaginal, gastric, kidney, and vascular systems.

In a preferred manner of use (e.g. cardiac, vascular, esophageal, gastric or rectal use), the system of the present invention may be used together with an ultrasound probe (either external, internal or percutaneous probe) operating at an appropriate frequency, that will ensure good visualization of the echo-opaque system components.

In the description that follows, a preferred embodiment of the device of the present invention that is suitable for intra-cardiac use will be described in more detail.

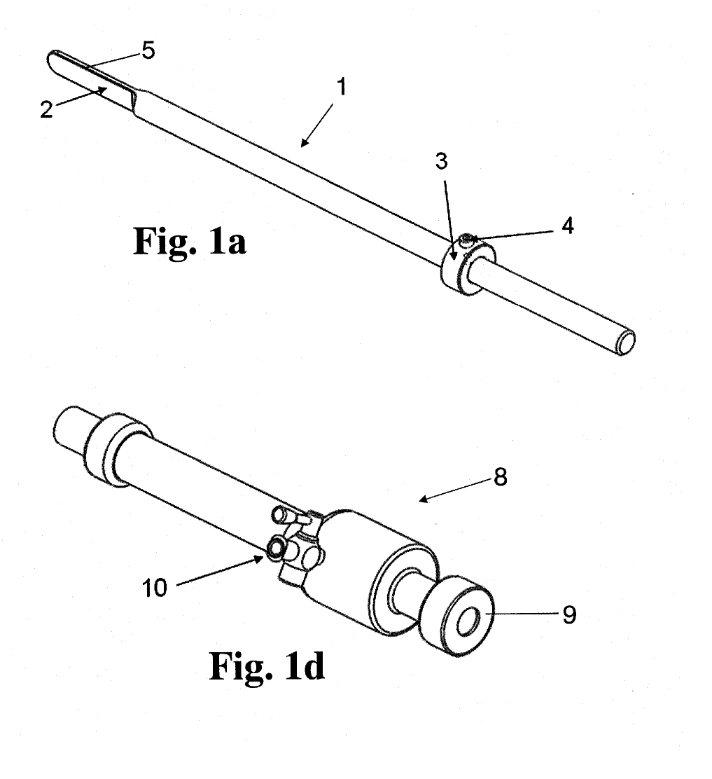

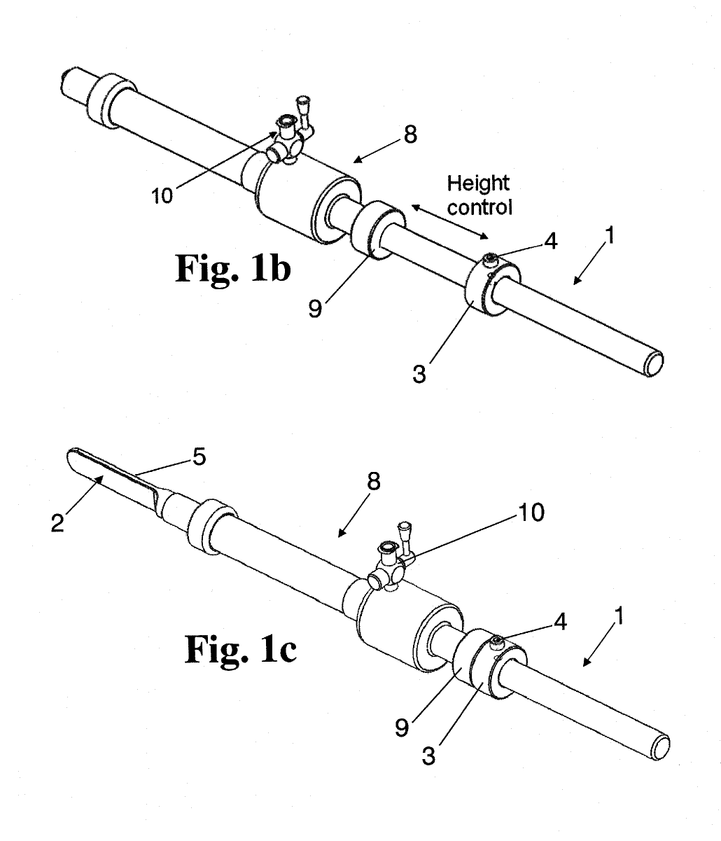

Thus, FIGS. 1a-1c describe one embodiment of the device of the present invention and its integration with a trocar/introducer sheath, or specifically, a trans-apical sheath (TAS), when used in a trans-apical delivery procedure. FIG. 1a describes an instrument referred to as a Papillary Muscle Pointer (PMP) 1 that may be used for pointing to (i.e. indicating the location of) the space between papillary muscles of a heart ventricle, and specifically those of the left ventricle. As may be seen in this figure, the PMP 1 is constructed in the form of an elongate rod having a thin rectangular-shaped blade 2 at a first, distal, end, and a rear stopper 3 with an integrated indicator element 4 towards its second, proximal end. Preferably, the indicator element 4 and one of the two side tips 5 of the blade 2 are aligned in parallel on the same angle of the rotational axes surrounding the PMP length axis. The indicator element 4 is preferably on the stopper. Optionally the indicator element 4 connects the stopper 3 to the PMP 1 body. It is to be noted that in the description and figures that follow, the term “indicator element” is used interchangeably with the term “sight”. The indicator element can also be a certain mark on the PMP proximal portion.

FIG. 1d illustrates an embodiment of a TAS 8. FIG. 1b depicts the insertion of the PMP 1 into a TAS 8. The PMP stopper 3 sets the limit for the PMP insertion depth/height, which are displayed on the PMP rod e.g. by using scale marks. The stopper 3 can be moved distally/proximally and fixed at the desired point such that the desired insertion length is met. The TAS 8 also comprises a rotatable indicator element 10 (also interchangeably referred to as “sight”). The sight 10 can be fixed to the TAS 8 body, such that the it is rotated along with the TAS 8 or it could be fixed such that it is rotationally moved in relation to the TAS body.

FIG. 1c shows the PMP 1 fully inserted into the TAS 5, until the PMP stopper 1b has reached the TAS inlet 9.

In general, the PMP 1 is inserted through the TAS 8 into the heart (in trans-apical approach). The PMP 1 and TAS 8 are aligned in the same rotational orientation such that the TAS sight 10 and the PMP sight 4 are parallel one to the other. An echocardiographic ultrasound probe test is performed, in order to achieve a short-axis view of the heart ventricles, in a height in which the papillary muscles of the ventricle, as well as an acoustic shadow of the PMP blade 2 are observed. One preferred view is achieved at the mid-papillary level, however additional slightly higher (between mid- and end-papillary level), or slightly lower (between mid- to sub-papillary level), may be used as well. The shadow features are in direct consequence of the PMP blade shape, depth, positioning and orientation. The papillary muscles are also viewed. The PMP 1 is rotated until being in a position vertically parallel between the papillary muscles, i.e. until the result acoustic shadow trail of the blade 2 is shown in a narrow manner between the acoustic images of the papillary muscles. This indicates that the blade 2 is indeed in the right orientation indicating the gap between the papillary muscles and substantially parallel to them. The TAS sight 10 is then rotated to be in the same rotational angle where the blade side edge 5 (and sight 4) is placed indicating the direction of the space (gap) between the papillary muscles. The PMP 1 is then retrieved. A desired delivery tool/device is inserted into the TAS 8 and is rotated and positioned in the direction and orientation according to sight 10 such that the tool/device or element of the tool/device is placed in between the papillary muscles gap when inserted therein A method for inserting an implant though a delivery tool in a desired orientation is disclosed in co-owned, co-pending International Patent Application no. PCT/IL09/00988 (published as WO 2010/046895) and incorporated herein by reference.

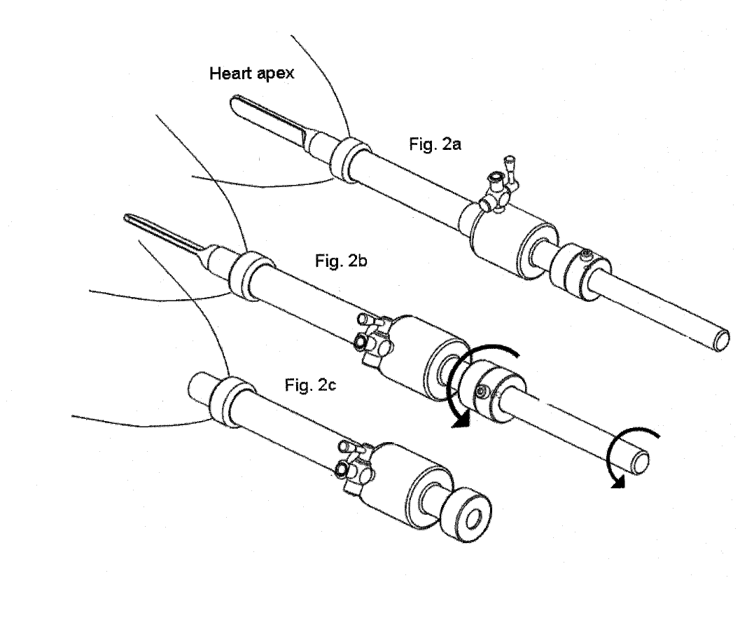

FIGS. 2a-2c demonstrate the use of the PMP 1 and the TAS 8 for orientation control. The orientation control can be used for Papillary Muscle gap pointing.

FIG. 2a shows a fully inserted PMP 1 inside the TAS 8, wherein both the TAS rotatable indicator element 10 and the corresponding element on the PMP (rotatable indicator element 4) are parallel and aligned.

FIG. 2b depicts the situation in which both PMP 1 and TAS 8 are rotated until the appropriate orientation is reached (i.e. according to an acoustic shade of the PMP blade indicating the gap between the papillary muscles, is seen during echocardiography). The PMP is rotated until the desired position is shown in the imaging system (i.e., until the narrow side of the blade is seen pointing at the desired anatomic direction, and the blade's acoustic trail is seen to originate from the center of the organ cavity). The TAS sight 10 is then rotated and aligned with the PMP sight 4 (optionally they can both be moved together when aligned). The PMP 1 can then be withdrawn, as seen in FIG. 2c, while maintaining the orientation and position of the TAS 8, whose rotatable indicator element 10 is now orientated in the inter-papillary muscle plane, and is now ready for insertion of the device delivery system, in which the device is loaded in a way that one of its arms is intended for positioning between the papillary muscles.

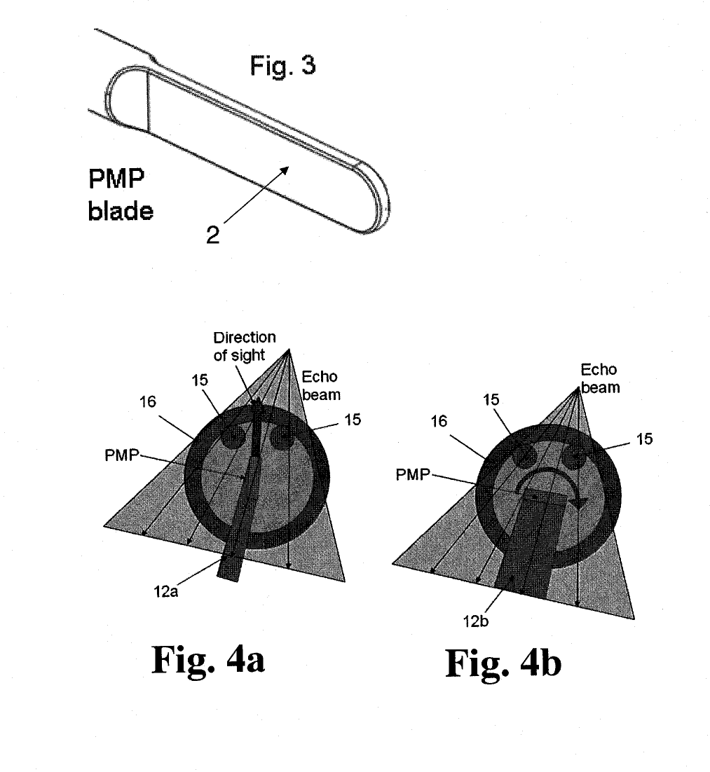

FIG. 3 provides a close-up view of one embodiment of the PMP's blade shape. The blade 2 shape is flat thus when being imaged the wide flat position can be seen and distinguished from the narrow position when rotated.

FIGS. 4a and 4b depict transverse sections of the left ventricle (viewed as 16), as typically acquired and visualized in a short-axis echocardiographic view at the mid-papillary level. The two round circles 15 labeled as papillary muscles (PM) represent the anterior and posterior papillary muscles of the left ventricle. In FIG. 4a, the PMP casts a narrow shadow trail 12a indicating the gap between the papillary muscles. It should be noted that in FIGS. 4a-4b the shadow of the PMP shows that the PMP is not physically in between the papillary muscles but at a distance from them (e.g. in the center of the ventricle). This is in order to clearly differentiate between the anatomy and the PMP, but more importantly, for correct centered positioning of the delivery and implantable device in the cavity of the ventricle. This could also be advantageous (when desired), that after the pointer is retrieved the implant is inserted at the location where the blade was positioned.

FIG. 4b shows a 90 degrees rotation of the PMP (in relation to the orientation of FIG. 4a) and it casts a broader shadow trail 12b between the papillary muscles, with respect to the PMP's blade shape depicted in FIG. 3.

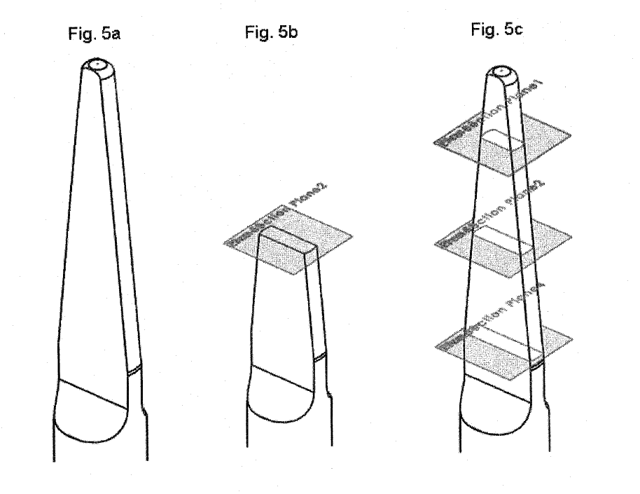

FIG. 5a depicts another optional configuration of the PMP blade 2, in which the transverse cross-sectional width and area changes decreasingly, gradually from bottom to top (from the proximal end of the blade to the distal end). FIG. 5b presents one of these aforementioned cross-sections, and FIG. 5c demonstrates the difference in width created for each cross-section, depending on the cross-section height. The length of blade is preferably 10-60 mm, the diameter of the rod is preferably 2-20 mm, the width of the blade is preferably 1-20 mm, the thickness of the blade is preferably 1-5 mm

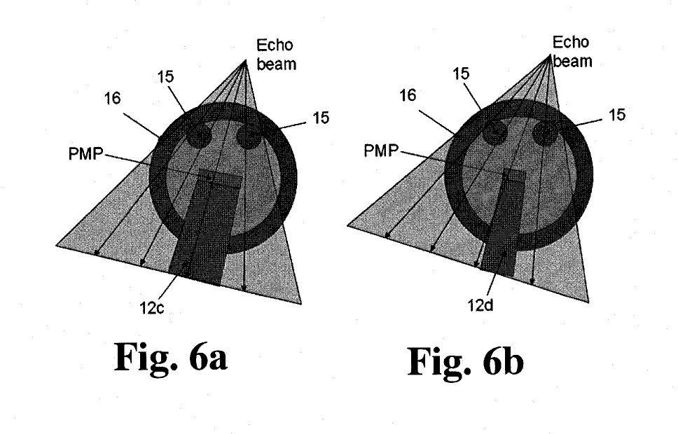

FIGS. 6a and 6b demonstrate how the PMP's tapered cross-sectional shape is used, in order to interpret the PMP blade acoustic shadow width, to height of PMP inside the left ventricle. 12c indicates a wide echo shadow and 12d shows a thinner echo shadow indicating the vertical positioning of the PMP blade.

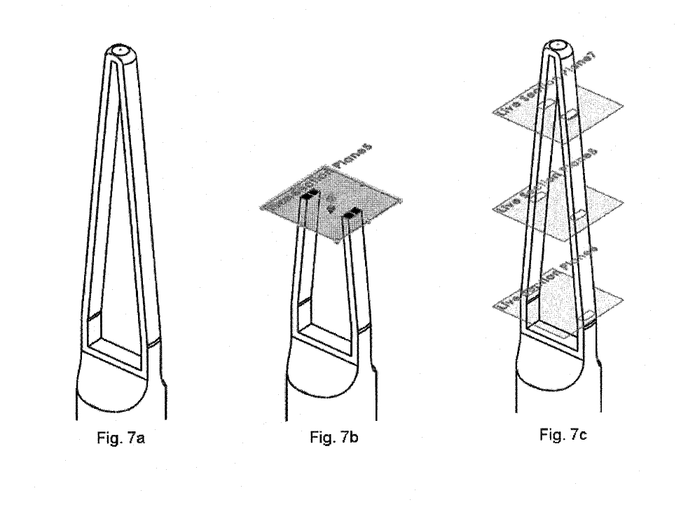

FIGS. 7a-7c depict an additional optional design for the PMP blade, enabling better echocardiographic resolution with fewer interruptions, as well as improved measuring and calibration capabilities. This is achieved by using a hollow blade contour. The blade is provided in the form of a hollow frame. FIG. 7a provides a close-up view of the PMP blade, while FIG. 7b provides a close-up view of a longitudinal cross-section of said blade, and FIG. 7c shows how various cross-sections in different heights, create different acoustic shadows of the PMP blade.

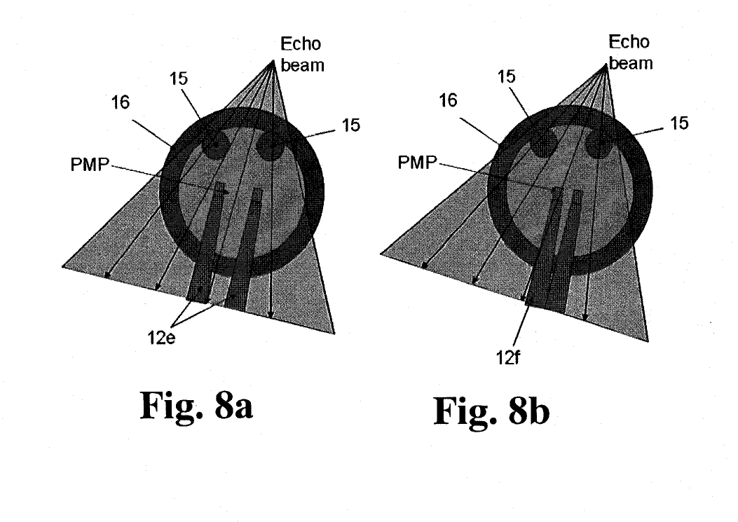

FIGS. 8a and 8b depict transverse sections of the left ventricle demonstrating the use of the hollow-blade type PMP. In FIG. 8a, a section of the hollow blade PMP's broad shadow 12e is projected between the papillary muscles. Following 90 degrees rotation of the PMP, it projects a narrower shadow (viewing only one shadow without seeing the space between the edges of the blade) between the papillary muscles. FIG. 8b shows the hollow blade wide portion 12f inserted in a lesser insertion depth than that as in FIG. 8a. Thus, the distance between the papillary muscles can be also estimated and verified using this embodiment of the PMP blade.

The space between the trails can be measured on echo cardiac imaging device since the geometry of the triangle (FIG. 7) is known the insertion depth can be calculated (by calculation, table, etc.) in relation to the entry length of the rod within the TAS. It can be used for data analysis, reference of dimensions, measurements of PM height within the heart, etc.

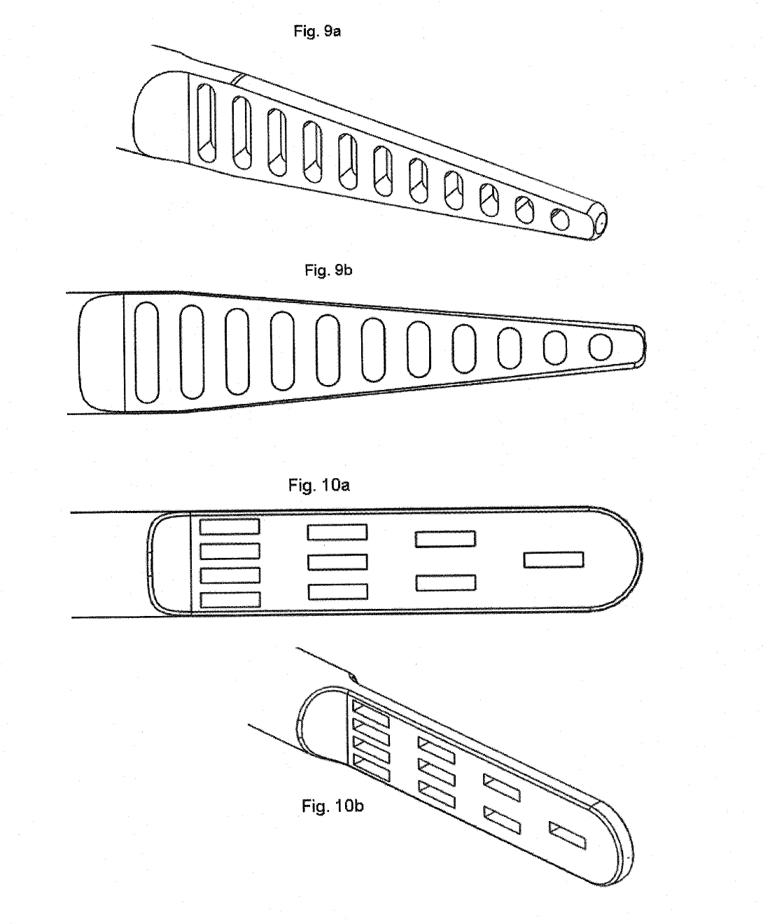

FIGS. 9a and 9b are enlarged views of an additional embodiment of the PMP blade, having a series of hollow contours, with decreasing gradual changing volume from bottom to top (from the proximal end to the distal end). A resolution can be set between the insertion depth and the count of the hollow contours as known to a person skilled in the art. Preferably, each contour gives an increment in progression of 1-3 mm.

FIGS. 10a and 10b provide close-up views of an additional possible configuration of the PMP blade, having a series of hollow contours, with decreasing gradual changing number of hollow elements from bottom to top (from the proximal end to the distal end). Hence, the number of acoustic shadows created and seen by ultrasound, can be used to assess the depth of insertion of the PMP. A resolution can be set between the insertion depth and the count of the hollow contours similarly as described before. In this case, the numbers of width contours shown indicate the insertion depth.

FIGS. 11a and 11b depict an embodiment in which a PMP and a Dilator are combined in one tool in order to enable a smooth and a-traumatic penetration into the left ventricle. When the proximal button 20 is pulled backwards, proximally towards the user, the tool can be used as a dilator (e.g. for insertion into trocar/introducer sheath, as a preliminary step for the implantation of intra-cardiac devices (such as those disclosed in co-owned, co-pending International Patent Application no. PCT/IL09/00988 [published as WO 2010/046895]). By pulling the button 20 forward, distally, an internal blade is exposed through a slit, thereby enabling the tool to function also as a PMP, having the same features as previously explained, while simplifying and shortening the procedure. The integrated Dilator-PMP is adapted to be inserted through the TAS. Similarly, the PMP and TAS can be combined in one tool. Additionally, the PMP can be combined in one tool with the delivery tool of the device intended for implantation.

According to an embodiment of the present invention, the dilator distal end has a conical structure. The conical structure is formed by two half cones wherein when the button (similarly to the button of the embodiment above) is pushed distally one half cone moves distally and acts as the blade (in the same manner as the blade of previous embodiments) when the button is pulled back proximally the half cone moves proximally again to form the conical structure.

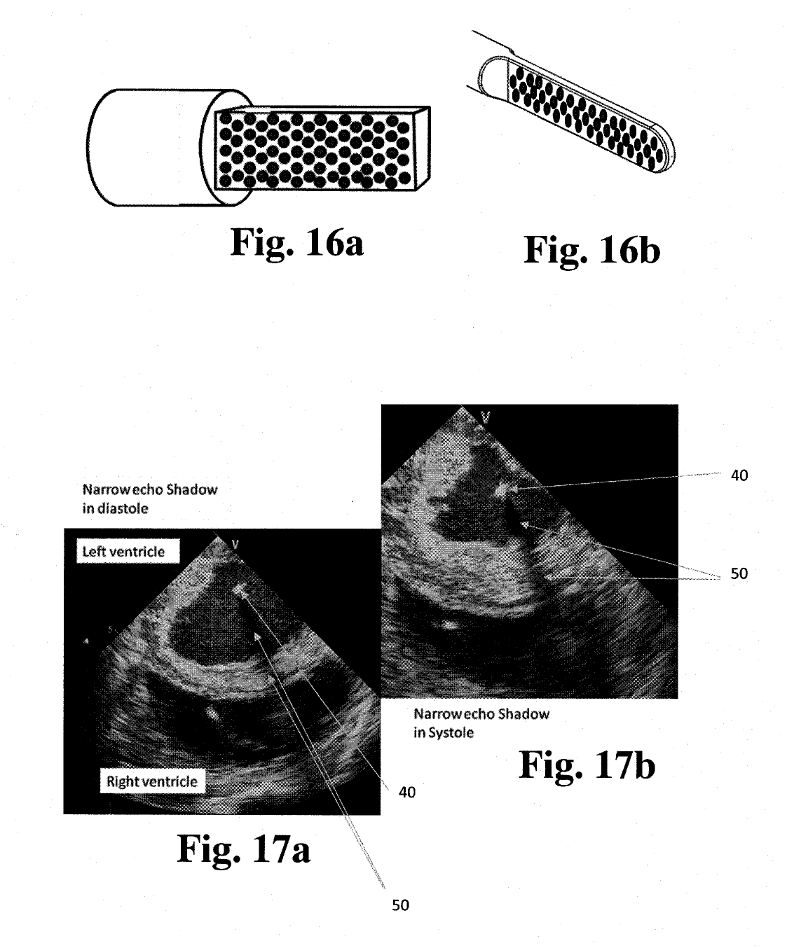

In one embodiment of the presently-disclosed device, bubbles containing air, oil or other echo-detectable fluid (having different acoustic impedance than that of the tissue or body fluid being screened) are incorporated within said device, in order to enhance acoustic contrast and thereby assist in visualization of the orientation of the device. FIGS. 16a-16b show examples of such embodiments wherein the blade comprises the bubbles.

In other versions of the device, various modifications may be made to the basic structure, in order to provide improved visualization characteristics. In one such embodiment, the device may be manufactured from two or more different materials having different acoustic impedance in order to enhance imaging contrast. In another embodiment, the acoustic impedance of different portions of the device (e.g. one or other of the blade two flat sides) may be enhanced by means of sand-blasting or gritting the selected surface. In another embodiment, the acoustic impedance of different portions of the device may also be enhanced by using different echogenic coatings in desired portions of the device.

In order to facilitate the use of the device of the present invention in a clinical environment, said device may form part of an integrated system which further comprises dedicated control and/or image-processing software, for further design and control of implantation route, and for fine-tuning of the positioning of the device. This aspect of the system of the present invention may also include an encoder or any additional incremental sensor, which will be able to accurately display the insertion height of the device. Such software can also “lock on” the specific defined points and track their movement in real time, in order to obtain data of orientation, depth, etc. (for example—data relevant to the space between the papillary muscles, the distance/orientation of the PMP shadow from the papillary muscles, etc.).

As described hereinabove, the elongate device (PMP) of the present invention may be constructed of one or more echo-opaque materials such as polymers (Acetal or Delrin, FEP, PTFE, Silicon,) or metals (such as stainless steel, gold, platinum, and cobalt-chromium alloys).

The device may be constructed using any of the suitable conventional manufacturing techniques that are well-known to the skilled artisan in the field, including (but not limited to) machining, casting, milling, extrusion, injection molding, stereolithography, sand blasting, grinding, polishing, and so on.

Different versions of the device that are intended for different clinical situations may be manufactured in a variety of different sizes. In the case of the PMP intended for intra-cardiac use (as described in more detail hereinabove), the length of the PMP is preferably in the range of 10 to 120 cm, the external diameter of the shaft is preferably in the range of 2 to 20 mm, the width of the distal blade is preferably in the range of 5 to 40 mm and the thickness of said blade is preferably in the range of 1 to 5 mm. As described hereinabove, the TAS device of the present invention may be constructed of one or more echo-opaque materials such as polymers (Acetal or Delrin, FEP, PTFE, Silicon,) or metals (such as stainless steel, gold, platinum, etc.).

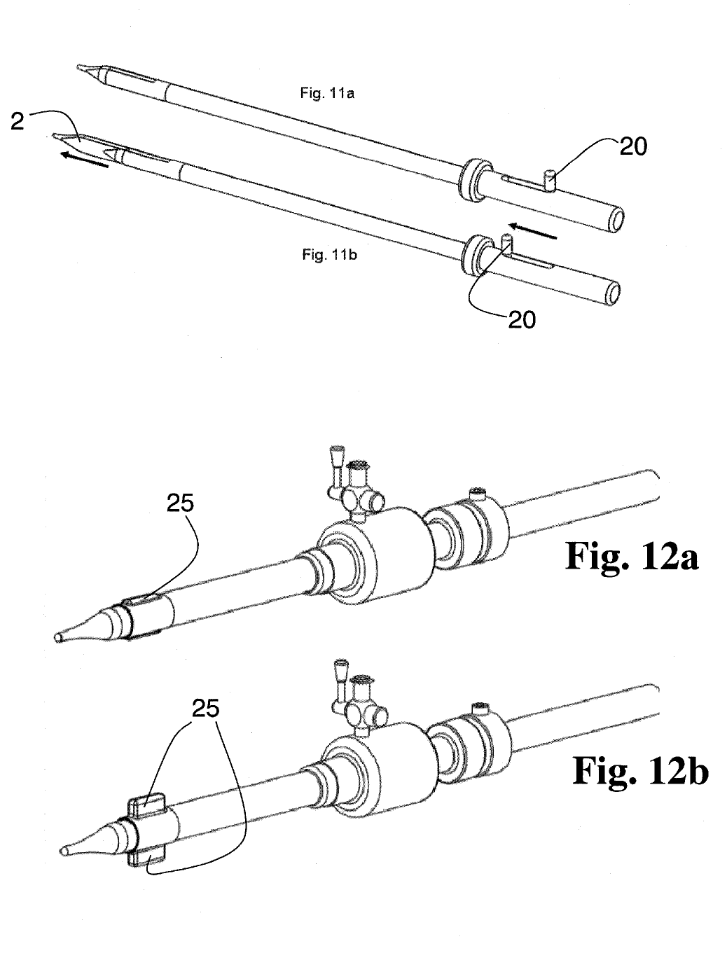

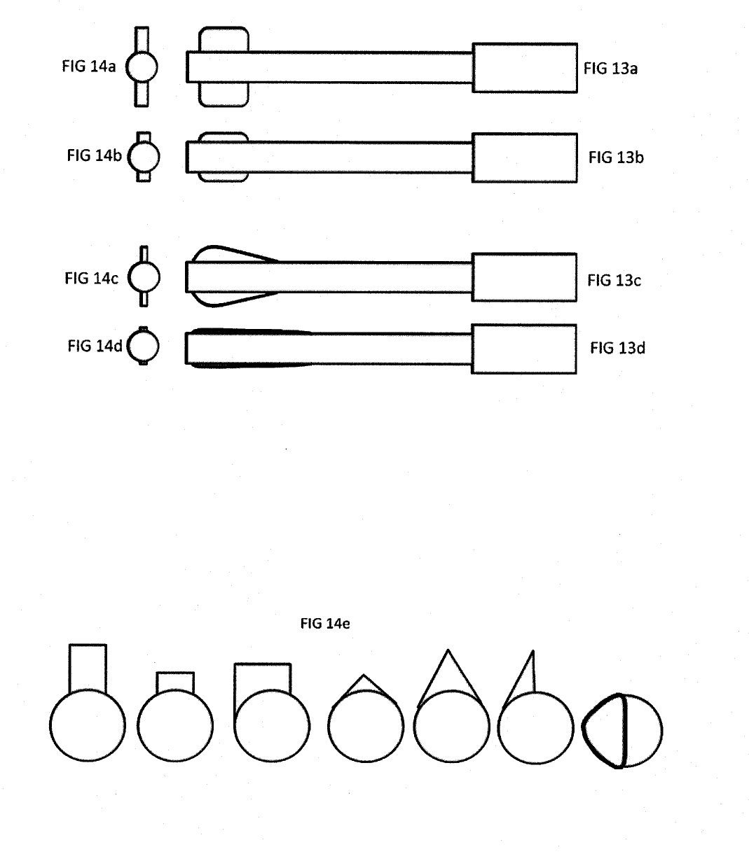

According to another embodiment of the present invention, the PMP comprises an inflatable balloon 25 as shown in FIGS. 12a-12b. The PMP is integrated with a dilator. When the balloon is inflated, it can be used as a pointer similarly to the manner of which the blade is used (described in previous embodiments). The inflated balloon gives a similar effect as the blade (e.g. of the embodiment described in FIG. 1a) having a wide orientation, and a narrow orientation when rotated. FIG. 12a shows the balloon in a deflated mode and FIG. 12a shows the balloon in an inflated mode. The balloon can be comprised of polyurethane, Dacron, nylon or other biocompatible polymers used for making these types of balloons. FIGS. 13a-13d illustrate examples of the inflated balloon shapes and FIGS. 14a-14d illustrate the cross sections of the inflated balloon shapes respectively. Preferably, the PMP comprises two inflatable balloons near its distal end opposite of each other such that when they are inflated they create a substantial flat mutual surface.

Optionally an extension such as a wire, spring, or NITI with a sheet covering can be used instead of the blade (and instead of the inflated balloon) for pointing and viewing the acoustic shade. The shape created has a flat surface similar to a blade and gives a clear narrow/wide shadow (depending on rotational orientation) when being imaged and therefore serves as an efficient pointer.

FIG. 14e illustrates several examples of cross-section shapes of the balloons attached to the PMP wherein said shapes are not necessarily symmetrical. The unsymmetrical balloon shapes enable to view the exact orientation angle, for example, the angle of 90 degrees and the angle of 270 degrees would give a different visual turnout during imaging.

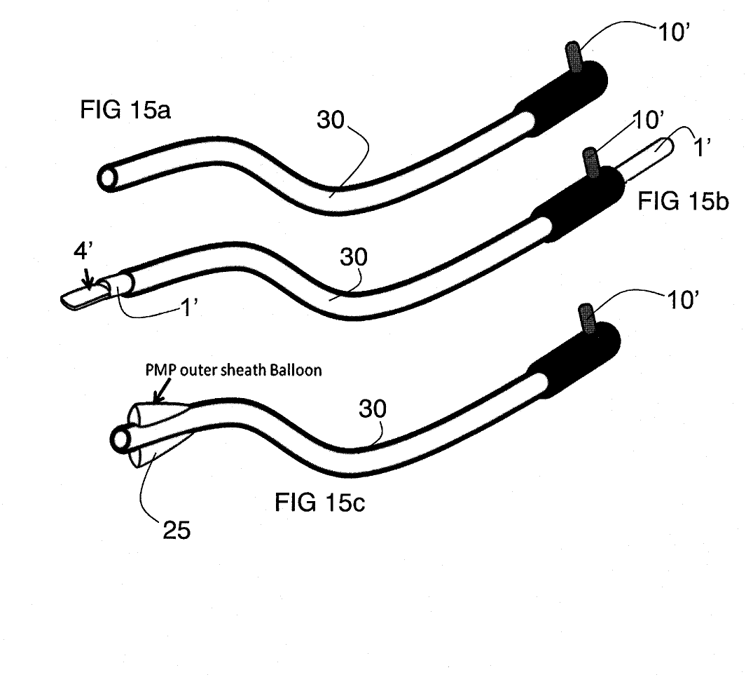

According to an embodiment of the present invention, the PMP is flexible, preferably comprised of plastic or Nitinol. It can be tubular or comprise a torque cable and adapted to enter a delivery tool such as a catheter. FIG. 15a illustrates a catheter tube 30 with a sight 10′ attached to it. FIG. 15b illustrates the catheter 30 with the PMP inside said catheter tube. The blade 4′ extends from the distal side of the PMP. The PMP 1′ comprises a sight (not shown) on its proximal end, aligned in the same orientation as one edge of blade 4′ (in a similar manner as explained in the embodiments hereinabove). When the proximal portion of PMP 1′ is rotated, the distal portion also rotates accordingly. After the PMP blade 4′ is correctly positioned, sight 10′ is aligned with the PMP sight. The PMP 1′ is then retracted and an implant/device is inserted in the orientation of sight 10′, (in a similar manner as explained in the embodiments hereinabove).

FIG. 15c illustrates another embodiment of the present invention wherein the delivery tool (e.g. working channel, catheter with or without an additional distal dilator, implant or instrument delivery system, etc.) comprises an inflatable balloon on its distal end. Catheter 30 comprises an inflatable fin like shaped balloons 25, one opposite of the other, on its distal end and acts as a pointer blade. The inflated balloons create a flat surface similar to a blade and give a clear narrow/wide shadow (depending on rotational orientation) when being imaged and therefore serves as an efficient pointer. The sight 10′ is aligned with one of the balloon edges. Optionally, also in this embodiment an extension such as a wire, spring, or NITI with a sheet covering can be used instead of the inflated balloon for pointing and viewing the acoustic shade.

The use of the preferred embodiment of the device of the invention disclosed hereinabove will now be described in relation to a procedure for the implantation of the three-armed “CORolla™” spring-like device (disclosed and claimed in co-pending WO 2010/046895 (incorporated herein by reference), in which one of the three arms of said spring-like device is to be positioned between the two papillary muscles, and at the same time leaning (as well as the rest of the arms) on the endocardium wall.

In this exemplary use, the PMP is inserted through the introducer sheath (TAS)/trocar (see FIG. 2a), through (but not limited to) the heart apex (or its proximity) into the left ventricle. The PMP penetration depth into the TAS, is limited by the PMP stopper.

Then, the PMP blade is used (as previously described) along with Echocardiography, for indicating the direction and location of the papillary muscles (which can be seen during the echocardiographic imaging).

The PMP can be rotated to either side (clockwise/counterclockwise) together with the trans-apical sheath, until the desired orientation is achieved and confirmed with echocardiography. Then, the PMP can be retrieved, while the trans-apical sheath is held in its current orientation vector direction, such that its rotatable indicator element will indicate the direction intended for the implant's inter-papillary-muscle arm location. The device is then loaded into its designated delivery tool (e.g. as explained in co-owned, co-pending International Patent Application no. PCT/IL09/00988 (published as WO 2010/046895) and incorporated herein by reference), in a way that ensures that its inter-papillary arm will be aligned with the rotatable indicator element, and the implantation is completed. Using echocardiography, the inter-papillary arm positioning is then confirmed.

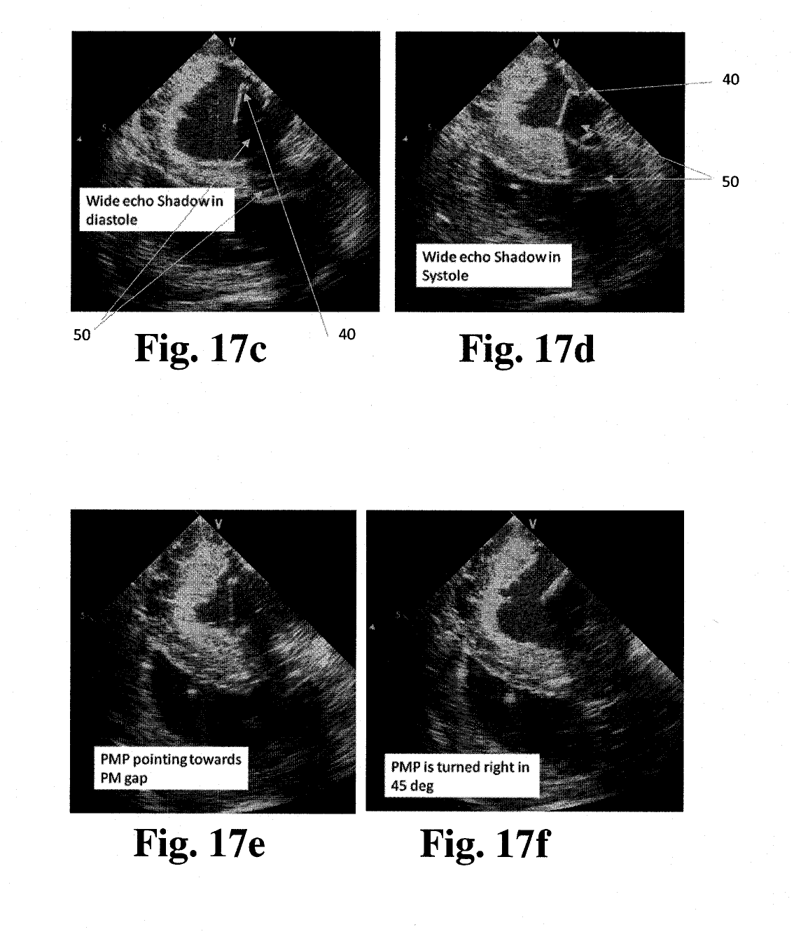

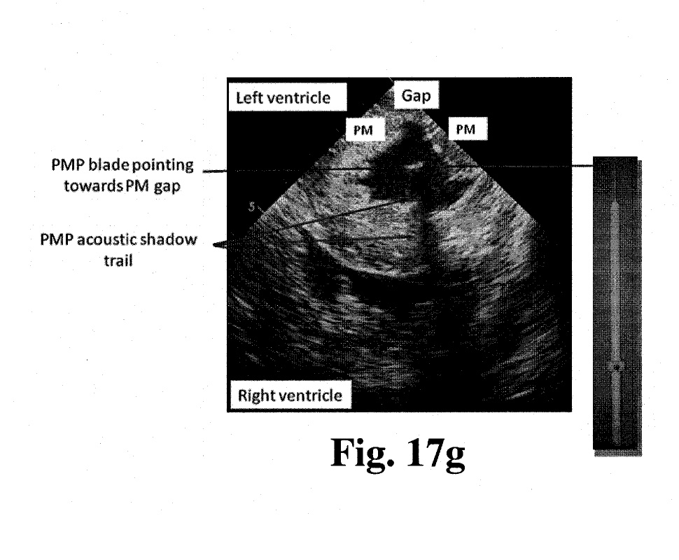

FIGS. 17a-17f illustrate different visual ultrasound images of the present invention blade inserted in the heart. FIG. 17a illustrates a narrow echo shadow, pointing to the inter-papillary muscle gap during diastole. Numeral 40 indicated the blade tip edge and 50 indicated portions of the shadow trail. FIG. 17b illustrates a narrow echo shadow during systole. FIG. 17c illustrates a wide echo shadow (formed due to rotation of 90 degrees of the blade) during diastole. FIG. 17d illustrates a wide echo shadow during systole. FIG. 17e illustrates the blade shadow pointing towards the papillary muscle gap. FIG. 17f illustrates the blade of FIG. 17e turned rightwards 45 degrees, giving a wider shadow.

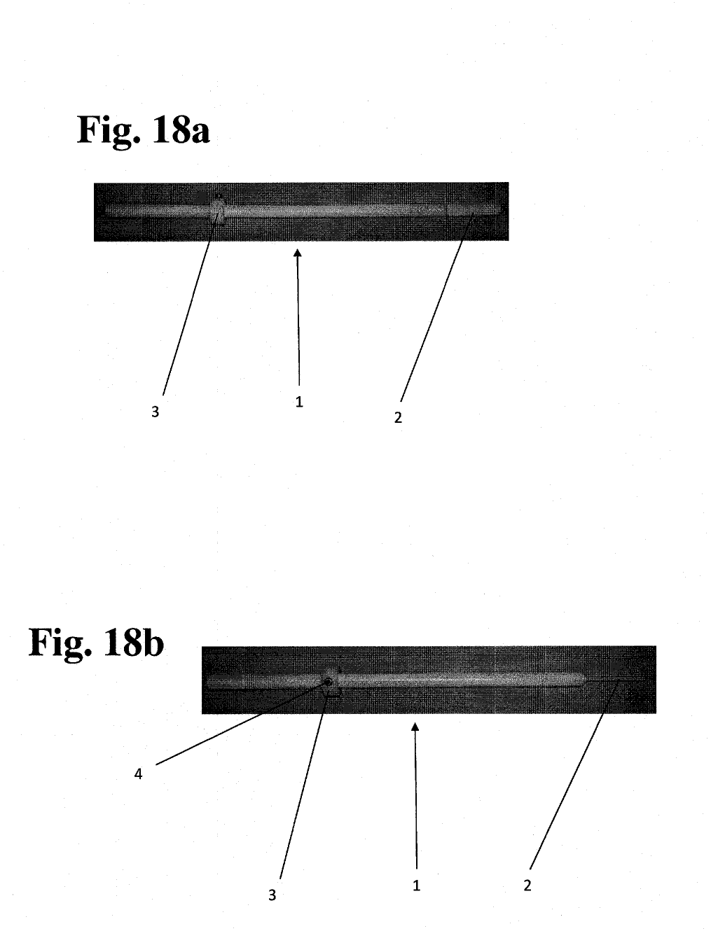

FIG. 17g illustrates a short-axis image of the blade shadow (in its narrow orientation) pointing towards the papillary muscle gap, indicating the acoustic shadow trail and the bade pointing toward the papillary muscle gap. It is clearly seen that the PMP edge is placed in the center of the ventricle. A pictorial view of the PMP (as shown in FIGS. 18a-18b) is shown on the right side.

FIGS. 18a-18b illustrate pictorial views of the PMP and its elements.

Thus the present invention indeed discloses a manner for clearly imaging the organs, while simultaneously imaging a designated pointer, and provides means for utilizing the data of the pointer images to then clearly precisely position an implants/devices within the organ. Also, according to a most preferred embodiment, the imaging is executed by using ultrasound alone.

While some of the embodiments of the invention have been described by way of illustration, it will be apparent that the invention can be carried into practice with many modifications, variations and adaptations, and with the use of numerous equivalents or alternative solutions that are within the scope of a person skilled in the art, without departing from the spirit of the invention, or the scope of the claims.

1. A system for assisting the placement of medical instruments, wherein said system comprises:
a. an elongate rod having a proximal end and a distal end, and wherein a blade is situated at said distal end and a rotational indicator element is placed close to the proximal end;
b. a sheath adapted to receive said rod therein comprising a rotational indicator element thereon.

2. The system of claim 1 wherein the rod further comprises a mechanical stopper element situated close to the proximal end of said rod.

3. The system of claim 2 wherein indicator element is placed on the mechanical stopper.

4. The system of claim 1 wherein the blade is elongated and substantially flat with a narrow thickness.

5. The system of claim 1 wherein the transverse cross-sectional width and area of the blade changes decreasingly, gradually from the proximal end of the blade to the distal end of the blade.

6. The system of claim 1 wherein the blade is provided in the form of a hollow frame.

7. The system of claim 5, wherein the blade comprises a series of hollow contours, with decreasing gradual changing volume of the contours from the proximal end to the distal end.

8. The system of claim 1 wherein the blade comprises a series of hollow contours, with decreasing gradual changing number of hollow contours from the proximal end to the distal end.

9. The system of claim 1, wherein the rod and sheath comprise one or more echo-opaque materials.

10. The system of claim 1, wherein the blade comprises bubbles wherein the bubbles material is selected from the group consisting of air, oil, and echo-detectable fluid.

11. The system of claim 1, further comprising a dilator, wherein the rod and a dilator are combined in one tool, wherein the combined tool is adapted to be inserted through the sheath.

12. The system of claim 11, wherein the combined tool comprises a button at its proximal end such that when said button is pushed distally the blade is exposed at the combined tool distal end and when said button is pulled proximally the blade enters the combined tool.

13. A system for assisting the placement of medical instruments, wherein said system comprises:
a. an elongate rod having a proximal end and a distal end and a rotational indicator element is placed close to the proximal end;
b. a dilator, wherein the rod and a dilator are combined in one tool, wherein the combined tool is adapted to be inserted through a sheath.
c. a sheath adapted to receive said combined tool therein comprising a rotational indicator element thereon.

14. The system of claim 13, wherein the combined tool further comprises two inflatable balloons near its distal end, opposite of each other such that when they are inflated they create a substantially flat mutual surface.

15. The system of claim 13, wherein the combined tool further comprises an inflatable balloon near its distal end.

16. The system of claim 15, wherein the inflatable balloon comprises an unsymmetrical shape when inflated.

17. The system of claim 13, wherein the dilator distal end has a conical structure and the conical structure is formed by two half cones; and wherein the combined tool comprises a button at its proximal end such that when said button is pushed distally one half cone moves distally and when said button is pulled proximally said one half cone moves proximally.

18. A system for assisting the placement of medical instruments, wherein said system comprises:
a. an elongate flexible rod having a proximal end and a distal end, and wherein a blade is situated at said distal end and a rotational indicator element is placed close to the proximal end;
b. a delivery tool adapted to receive said rod therein comprising a rotational indicator element thereon.

19. The system of claim 18, wherein the delivery tool is selected from the group consisting of working channel, implant delivery system, instrument delivery system, catheter, and catheter with an additional distal dilator.

20. A system for assisting the placement of medical instruments, wherein said system comprises:
a delivery tool comprising a rotational indicator element thereon and two inflatable balloons near its distal end, opposite of each other such that when they are inflated they create a substantially flat mutual surface aligned in the same rotational orientation as said indicator element.

21. A method for assisting in the correct placement of a medical instrument within a target tissue of a mammalian subject, comprising the steps of:
a) inserting a hollow sleeve fitted with a rotational indicator element into said target tissue;
b) providing an elongated rod having a blade-like structure at its distal end and a rotatable indicator element and stopper towards its proximal end;
adjusting the position of said stopper on said elongated rod such that a desired distance between said stopper and the distal tip of said rod is achieved;
c) inserting said elongated rod into the internal bore of said hollow sleeve until its stopper has reached the proximal end of said sleeve.
d) observing the position and orientation of said elongated rod by means of acoustic imaging;
e) rotating the elongated rod until the distal blade is in the desired rotational orientation;
f) adjusting the rotational indicator on the hollow sleeve until it is located parallel to the rotational indicator on the elongated rod;
g) removing said elongated rod; and
i) inserting a desired medical instrument into the internal bore of the hollow sleeve and rotating same until it is orientated in the desired rotational plane as indicated by the rotational indicator on said hollow sleeve.

22. The method according to claim 21, wherein the medical instrument is an intracardiac device and wherein the rotational adjustment in step (e) is made such that the blade of the elongated rod becomes orientated within the plane defined by the space between the ventricular papillary muscles.

23. The method according to claim 22, further comprising a step after step e, comprising positioning the blade distal portion such that said blade distal portion is placed in the center of the ventricle,

24. The method according to claim 23 wherein the placement of the distal blade in the center of the ventricle is implemented by viewing the acoustic trail casted by the pointer.

25. A method for assisting in the correct placement of a medical instrument within a target tissue of a mammalian subject, comprising the steps of:
a) inserting a delivery tool fitted with a rotational indicator element into said target tissue;
b) providing an elongated flexible rod having a blade-like structure at its distal end and a rotatable indicator element towards its proximal end; inserting said elongated flexible rod into the internal bore of said delivery tool.
c) observing the position and orientation of said elongated flexible rod by means of acoustic imaging;
d) rotating the elongated flexible rod until the distal blade is in the desired rotational orientation;
e) adjusting the rotational indicator on the delivery tool until it is located parallel to the rotational indicator on the elongated flexible rod;
f) removing said elongated flexible rod; and
g) inserting a desired medical instrument into the internal bore of the delivery tool and rotating same until it is orientated in the desired rotational plane as indicated by the rotational indicator on said delivery tool.

26. A method according to claim 24, wherein the delivery tool is selected from the group consisting of working channel, implant delivery system, instrument delivery system, catheter, and catheter with an additional distal dilator.

27. A method for assisting in the correct placement of a medical instrument within a target tissue of a mammalian subject, comprising the steps of:
a) inserting a delivery tool into said target tissue;
b) providing one or more inflatable balloons at said delivery tool distal end and a rotatable indicator element towards its proximal end aligned with said one or more inflatable balloons structure;
c) inflating said one or more inflatable balloons;
d) observing the position and orientation of said one or more inflatable balloons by means of acoustic imaging;
e) rotating the elongated flexible rod until one or more inflatable balloons structure is in the desired rotational orientation;
f) inserting a desired medical instrument into the internal bore of the delivery tool and rotating same until it is orientated in the desired rotational plane as indicated by the rotational indicator on said delivery tool.
